(19) United States
(12) Patent Application Publication
(10) Pub. No.: US 2019/0104924 A1
(43) Pub. Date: Apr. 11, 2019

(54) INSERTION APPARATUS

(71) Applicant: OLYMPUS CORPORATION, Tokyo (JP)

(72) Inventor: Yasuhiro OKAMOTO, Tokyo (JP)

(73) Assignee: OLYMPUS CORPORATION, Tokyo (JP)

(21) Appl. No.: 16/212,805

(22) Filed: Dec. 7, 2018

Related U.S. Application Data
(63) Continuation of application No. PCT/JP2017/008105, filed on Mar. 1, 2017.

(30) Foreign Application Priority Data
Aug. 2, 2016 (JP) 2016-152113

(51) Int. Cl. A61B 1/005; A61B 1/05; A61B 1/00; A61B 1/06

(52) U.S. Cl. CPC A61B 1/0055; A61B 1/05; A61B 1/0052; A61B 1/0661; A61B 1/00114

(57) ABSTRACT

An insertion apparatus includes: an insertion portion which is inserted into a body cavity of a subject and detachably equipped with a spiral tube rotatable around a longitudinal axis, the insertion portion having predetermined flexibility; and a driving source configured to cause the spiral tube to rotate; and the spiral tube is configured with a structure set not to bend at an arbitrary bending angle or more, the arbitrary bending angle being an angle at which rotation of the spiral tube is not stopped by driving force of the driving source even if the spiral tube receives external force to try to maintain a bending shape from a touched body cavity wall.

### CROSS REFERENCE TO RELATED APPLICATION

This application is a continuation application of PCT/JP2017/008105 filed on Mar. 1, 2017 and claims benefit of Japanese Application No. 2016-152113 filed in Japan on Aug. 2, 2016, the entire contents of which are incorporated herein by this reference.

### BACKGROUND OF THE INVENTION

### Field of the Invention

The present invention relates to an insertion apparatus having a driving source and a driven member arranged in a flexible tube and a transmitting member provided along a long axis in the flexible tube and configured to transmit rotational driving force of the driving source to the driven member.

### Description of the Related Art

Endoscopes are used in a medical field, an industrial field and the like.

Medical endoscopes can be used to perform observation, examination, treatment, or the like by inserting an insertion portion into a body cavity which is an examined part.

An endoscope generally has an insertion portion, an operation portion and a universal cord. In a configuration in which the insertion portion has a flexible tube portion, the insertion portion is inserted into a gastrointestinal tract, which is a body cavity, transanally, orally or nasally.

The endoscope is configured such that the flexible tube portion of the insertion portion is provided with a corrugated tube having flexibility, and, at the time of inserting the insertion portion having the flexible tube portion, for example, into an intestinal tract, a user causes the insertion portion to be inserted toward a deep part of the intestinal tract by performing a twisting operation or a feeding operation of the insertion portion located outside the body, while operating a bending operation knob provided on the operation portion to cause a bending portion to bend.

However, the twisting operation or the feeding operation, which is a technique for causing the insertion portion to be smoothly inserted toward a deep part of a body cavity, requires skill. Therefore, for the endoscope, an electric mechanism portion such as an insertion assisting mechanism for causing the insertion portion to move toward or away from a deep part is well known, for example, as disclosed in International Publication No. 2015-072233.

As for the insertion apparatus of International Publication No. 2015-072233, a configuration is disclosed in which a tube body having a corrugated tube and provided being extended in a longitudinal direction, a driving source disposed on a proximal end side of the tube body, a driven member disposed on a distal end side of the tube body, and a transmitting member provided along a long axis of the tube body in the tube body and configured to transmit rotation to the driven member by being rotated around the long axis by driving force of a motor or the like which is the driving source are provided.

As for the conventional insertion apparatus disclosed in International Publication No. 2015-072233, a technique is disclosed in which a rotation driving source or the driven member is arranged before the transmitting member configured to transmit rotation force of the rotation driving source which the electric mechanism portion is provided with to the driven member, without damaging a function of the electric mechanism portion, so that the tube body having the corrugated tube is prevented from being broken by twisting force from the rotation driving source or twisting force from the driven member.

### SUMMARY OF THE INVENTION

An insertion apparatus in an aspect of the present invention includes: an insertion portion which is inserted into a body cavity of a subject and provided with a cylindrical body rotatable around a longitudinal axis on an outer circumferential surface, the insertion portion having predetermined flexibility; and a driving source configured to cause the cylindrical body to rotate; wherein the cylindrical body is configured with a structure, the structure as a whole having predetermined bending rigidity and the structure being set not to bend at an arbitrary bending angle or more so that rotation by driving force of the driving source does not stop even if the structure receives external force to try to maintain a bending shape of the body cavity from a body cavity wall that the structure touches, due to the predetermined bending rigidity.

### BRIEF DESCRIPTION OF THE DRAWINGS

FIG. 1 is a diagram showing an endoscope apparatus, which is an insertion apparatus, according to an aspect of the present invention;

FIG. 2 is a diagram showing a configuration for transmitting rotational driving force to a rotation unit according to the aspect of the present invention;

FIG. 3 is a diagram showing a configuration of a bending portion, a first flexible tube portion, a second flexible tube portion and the rotation unit according to the aspect of the present invention;

FIG. 4 is a diagram showing a configuration of the second flexible tube portion, a third flexible tube portion, a base portion and the rotation unit according to the aspect of the present invention;

FIG. 5 is a cross-sectional view of a V-V line in FIG. 4 according to the aspect of the present invention;

FIG. 6 is an exploded perspective view in which the first flexible tube portion and the second flexible tube portion are disassembled in members according to the aspect of the present invention;

FIG. 7 is an exploded perspective view showing a first form of a spiral tube, in which a tube portion is disassembled in members, according to the aspect of the present invention;

FIG. 8 is a side view showing the rotation unit according to the aspect of the present invention;

FIG. 9 is a cross-sectional view of the tube portion according to the aspect of the present invention;

FIG. 10 is a side view showing a state in which an insertion portion provided with the rotation unit is bent, according to the aspect of the present invention;

FIG. 11 is a cross-sectional view of a corrugated tube which is bent, according to the aspect of the present invention;

FIG. 12 is a side view showing a second form of the spiral tube and showing the rotation unit, according to the aspect of the present invention;

FIG. 13 is a cross-sectional view of the tube portion according to the aspect of the present invention;

FIG. 14 is a side view showing a state in which the insertion portion provided with the rotation unit is bent, according to the aspect of the present invention;

FIG. 15 is a cross-sectional view of a helical tube which is bent, according to the aspect of the present invention;

FIG. 16 is a side view showing a third form of the spiral tube and showing the rotation unit, according to the aspect of the present invention;

FIG. 17 is a cross-sectional view of the tube portion according to the aspect of the present invention;

FIG. 18 is a side view showing a state in which the insertion portion provided with the rotation unit is bent, according to the aspect of the present invention;

FIG. 19 is a side view showing a first form of the second flexible tube portion and showing the second flexible tube portion equipped with the rotation unit, according to the aspect of the present invention;

FIG. 20 is a cross-sectional view of the second flexible tube portion according to the aspect of the present invention;

FIG. 21 is a side view showing a state in which the insertion portion provided with the rotation unit is bent, according to the aspect of the present invention;

FIG. 22 is a cross-sectional view of the helical tube which is bent, according to the aspect of the present invention;

FIG. 23 is a side view showing the second flexible tube portion equipped with the rotation unit of the second form of the spiral tube, according to the aspect of the present invention;

FIG. 24 is a cross-sectional view of the second flexible tube portion according to the aspect of the present invention;

FIG. 25 is a side view showing a state in which the second flexible tube portion equipped with the rotation unit is bent, according to the aspect of the present invention;

FIG. 26 is a cross-sectional view of the corrugated tube which is bent, according to the aspect of the present invention;

FIG. 27 is a side view showing a third form of the second flexible tube portion and showing the second flexible tube portion equipped with the rotation unit, according to the aspect of the present invention;

FIG. 28 is a cross-sectional view of the second flexible tube portion according to the aspect of the present invention; and

FIG. 29 is a side view showing a state in which the second flexible tube portion provided with the rotation unit is bent, according to the aspect of the present invention.

### DETAILED DESCRIPTION OF THE PREFERRED EMBODIMENT(S)

An embodiment of the present invention will be described below with reference to drawings.

Note that, on each of the drawings used in the description below, a reduced scale of each component may be caused to be different so that the component is in a size recognizable on the drawing. That is, the present invention is not limited only to the number of components, shapes of the components, a ratio of sizes of the components, relative positional relationships among the respective components shown in the drawings.

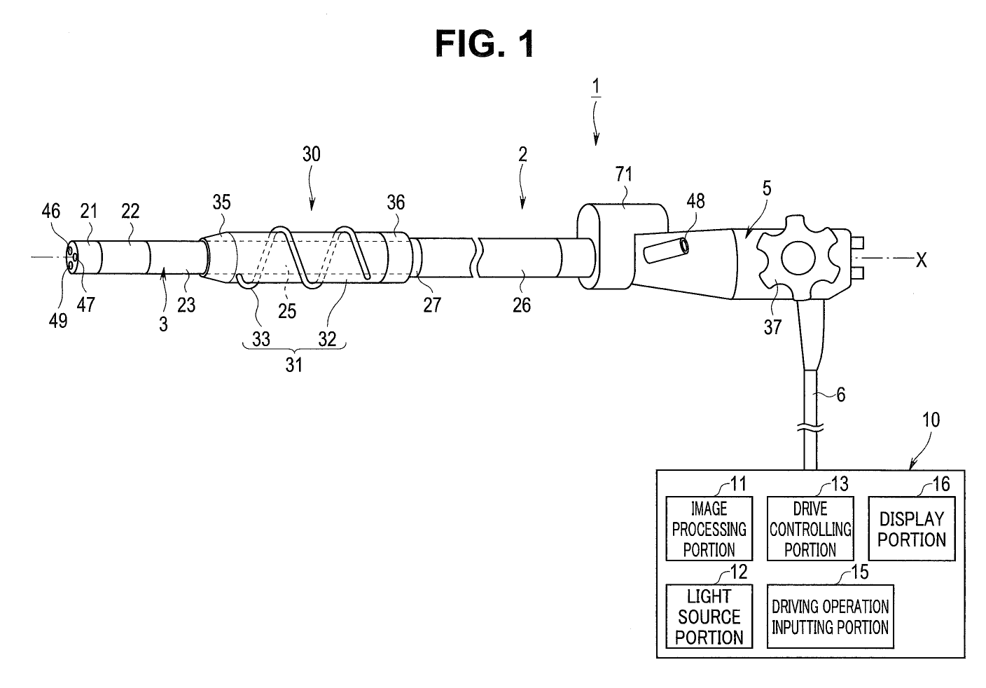

The embodiment of the present invention will be described with reference to FIGS. 1 to 6.

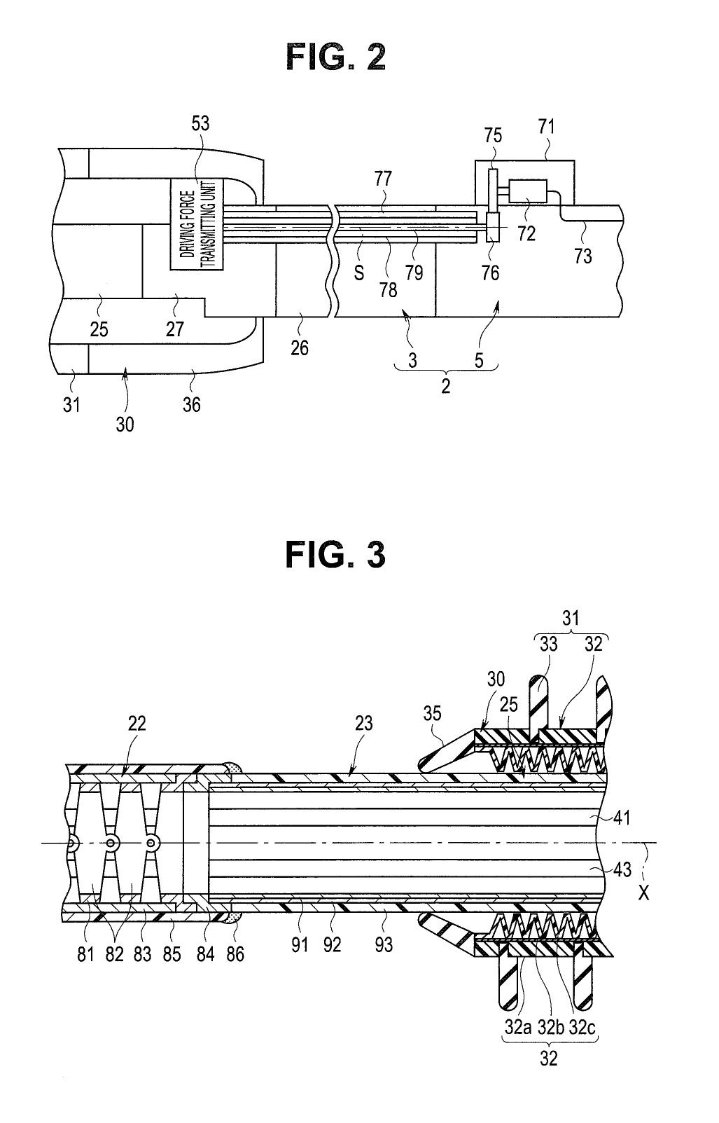

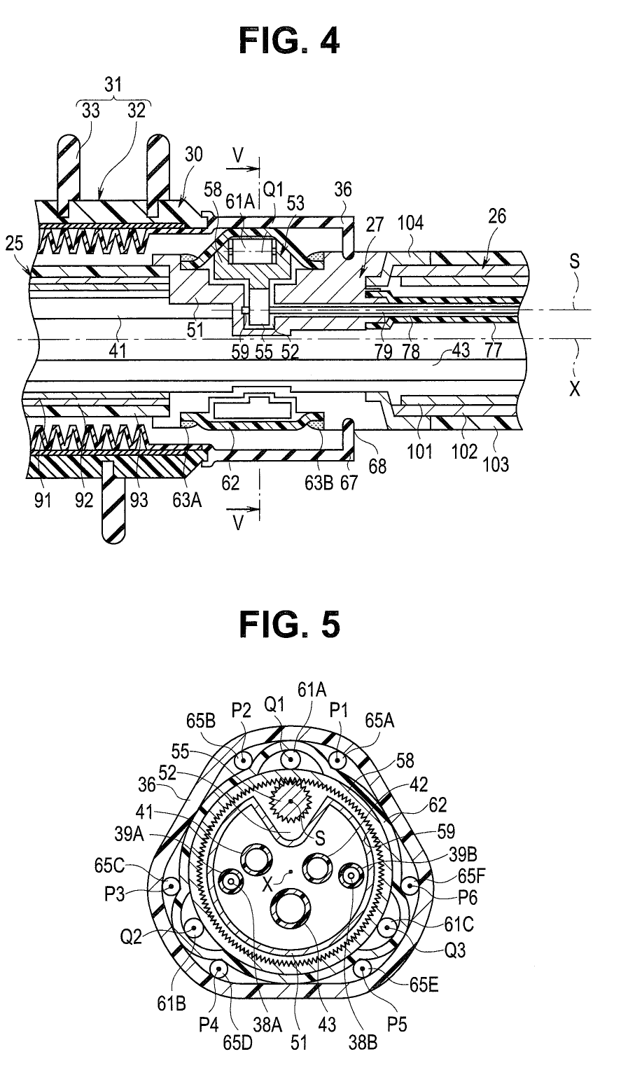

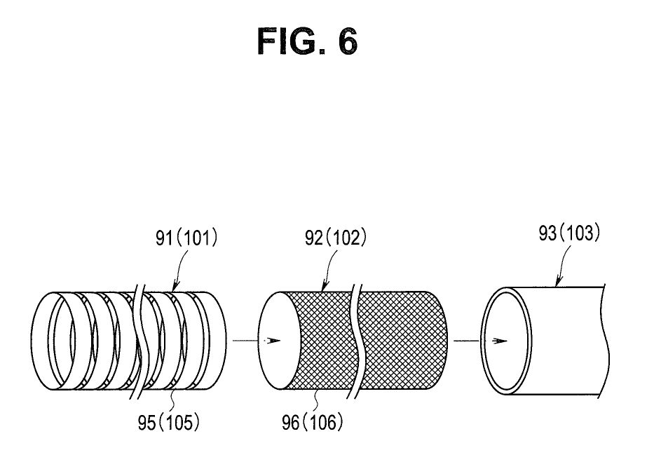

An embodiment of an endoscope apparatus, which is an insertion apparatus of an aspect of the present invention, will be described with reference to drawings. FIG. 1 is a diagram showing the endoscope apparatus which is an insertion apparatus; FIG. 2 is a diagram showing a configuration for transmitting rotational driving force to a rotation unit; FIG. 3 is a diagram showing a configuration of a bending portion, a first flexible tube portion, a second flexible tube portion and the rotation unit; FIG. 4 is a diagram showing a configuration of the second flexible tube portion, a third flexible tube portion, a base portion and the rotation unit; FIG. 5 is a cross-sectional view of a V-V line in FIG. 4; and FIG. 6 is an exploded perspective view in which the first flexible tube portion and the second flexible tube portion are disassembled in members.

As shown in FIG. 1, an endoscope apparatus 1 has a longitudinal axis X. Description will be made below on an assumption that an extension side of an insertion portion 3, which is one of directions parallel to the longitudinal axis X of the endoscope 2, is a proximal end direction, and an operation portion 5 side in a direction opposite to the proximal end direction is a distal end direction. The distal end direction and the proximal end direction are axial parallel directions which are parallel to the longitudinal axis X.

The endoscope apparatus 1 is provided with the endoscope 2 which is an insertion device. The endoscope 2 is provided with the insertion portion (the endoscope insertion portion) 3 provided being extended along the longitudinal axis X, an operation portion (an endoscope operation portion) 5 provided on a more proximal end direction side than the insertion portion 3, and a peripheral unit 10.

Note that the peripheral unit 10 is provided with an image processing portion 11 such as an image processor, a light source portion 12 provided with a light source such as a lamp, a drive controlling portion 13 which is a control device provided with, for example, a power source, a storage portion such as a memory, and a CPU or an ASIC, a driving operation inputting portion 15 which is a button, a foot switch or the like, and a display portion 16 such as a monitor.

The insertion portion 3 of the endoscope 2 is provided being extended along the longitudinal axis X and inserted into a body cavity when the endoscope apparatus 1 is used. The insertion portion 3 is provided with a distal end constituting portion 21 forming a distal end of the insertion portion 3, a bending portion 22 provided on a more proximal direction side than the distal end constituting portion 21, a first flexible tube portion 23 provided on a more proximal end direction side than the bending portion 22, a second flexible tube portion 25 provided on a more proximal end direction side than the first flexible tube portion 23, and a third flexible tube portion 26 provided on a more proximal end direction side than the second flexible tube portion 25.

Along the axial parallel direction parallel to the longitudinal axis X between the second flexible tube portion 25 and the third flexible tube portion 26, a base portion 27 is provided. The second flexible tube portion 25 is coupled with the third flexible tube portion 26 via the base portion 27.

Here, it is assumed that, on a section orthogonal to the longitudinal axis X, a direction away from the longitudinal axis X is an outer circumferential direction (an off-axis direction), and a central direction toward the longitudinal axis X is an inner circumferential direction (an inner axis direction).

The insertion portion 3 is provided with a rotation unit 30 in a cylindrical shape, which is of a disposable-type here, on the outer circumferential direction side. That is, the insertion portion 3 is in a state of being inserted in the rotation unit 30, and the rotation unit 30 is fitted to the second flexible tube portion 25.

In the endoscope 2, by rotational driving force being transmitted in the state that the rotation unit 30 is fitted to the insertion portion 3, the rotation unit 30 rotates around the longitudinal axis X relative to the insertion portion 3.

The rotation unit 30 is provided with a spiral tube 31 provided being extended along the longitudinal axis X. The spiral tube 31 is provided with a tube portion 32, a fin portion 33 provided being extended on an outer circumferential surface of the tube portion 32. Note that a configuration of the tube portion 32 will be described in detail later. Note that, as for the spiral tube 31, the tube portion 32 itself may be a corrugated tube.

The fin portion 33 is provided being extended from the proximal end direction toward the distal end direction, in a helical shape with the longitudinal axis X as a center. On the distal end direction side of the spiral tube 31 of the rotation unit 30, a distal end side cylindrical portion 35 is provided.

The distal end side cylindrical portion 35 is formed in such a tapered shape that an outer diameter gradually decreases toward the distal end direction side. On the proximal end direction side of the spiral tube 31, a proximal end side cylindrical portion 36 in a cylindrical shape is provided.

By the rotation unit 30 rotating around the longitudinal axis X in a state in which the fin portion 33 of the spiral tube 31 is pressed in the inner circumferential direction by a body cavity wall or the like, thrust in the distal end direction or the proximal end direction acts on the insertion portion 3 and the rotation unit 30.

That is, movability of the insertion portion 3 in an insertion direction (the distal end direction) in a body cavity such as an inside of a small intestine or an inside of a large intestine is improved by the thrust in the distal end direction, and movability of the insertion portion 3 in a removal direction (the proximal end direction) in the body cavity is improved by the thrust in the proximal end direction.

One end of a universal cord 6 is connected to the operation portion 5 of the endoscope 2. The other end of the universal cord 6 is connected to the peripheral unit 10. On an outer surface of the operation portion 5, a bending operation knob 37 to which a bending operation of the bending portion 22 is inputted is provided.

On the outer surface of the operation portion 5, a treatment instrument insertion portion 48 into which a treatment instrument such as forceps is inserted is provided. The treatment instrument insertion portion 48 communicates with a channel tube 43 (see FIG. 3) disposed in the insertion portion 3.

That is, one end of the channel tube 43 is connected to the treatment instrument insertion portion 48 through an inside of the insertion portion 3 and an inside of the operation portion 5. A treatment instrument inserted from the treatment instrument insertion portion 48 projects in the distal end direction from an opening portion 49 of the distal end constituting portion 21 through an inside of the channel tube 43. Then, treatment by the treatment instrument is performed in the state in which the treatment instrument projects from the opening portion 49 of the distal end constituting portion 21.

A motor housing 71 is coupled with the operation portion 5. A motor 72 (see FIG. 2), which is a driving source, is accommodated inside the motor housing 71.

As shown in FIG. 2, one end of a motor cable 73 is connected to the motor 72 accommodated in the motor housing 71 provided in the operation portion 5. The motor cable 73 is provided being extended through the inside of the operation portion 5 and an inside of the universal cord 6, and the other end is connected to the drive controlling portion 13 of the peripheral unit 10.

The motor 72 is driven by electric power being supplied from the drive controlling portion 13 via the motor cable 73. Then, by the motor 72 being driven, rotational driving force to cause the rotation unit 30 to rotate is generated. A relay gear 75 is fitted to the motor 72. Inside the operation portion 5, a driving gear 76 to engage with the relay gear 75 is provided.

As shown in FIG. 3, inside the insertion portion 3, an image pickup cable 41, a light guide 42 and the channel tube 43 described above are provided being extended along the longitudinal axis X.

The bending portion 22 of the insertion portion 3 is provided with a bending tube 81. The bending tube 81 is provided with a plurality of bending pieces 82 made of metal.

Each of the bending pieces 82 is rotatably coupled with adjoining bending pieces 82. In the bending portion 22, the outer circumferential direction side of the bending tube 81 is covered with a bending mesh tube 83, which is a bending braid. The bending mesh tube 83 is formed by metallic strands (not shown) woven in mesh.

In the bending portion 22, the outer circumferential direction side of the bending mesh tube 83 is covered with a bending outer cover 85. The bending outer cover 85 is formed, for example, by fluorocarbon rubber.

Inside the distal end constituting portion 21 (a distal end portion) of the insertion portion 3, an image pickup device (not shown) configured to pick up an image of an object is provided. The image pickup device picks up an image of an object through an observation window 46 provided on the distal end constituting portion 21 of the endoscope 2 shown in FIG. 1.

One end of the image pickup cable 41 is connected to the image pickup device. The image pickup cable 41 is provided being extended through the inside of the insertion portion 3, the inside of the operation portion 5 and the inside of the universal cord 6, and the other end is connected to the image processing portion 11 of the peripheral unit 10 shown in FIG. 1.

Image processing of a picked-up object image is performed by the image processing portion 11 to generate an image of an object. Then, the generated image of the object is displayed on the display portion 16 (see FIG. 1).

The light guide 42 is provided being extended through the inside of the insertion portion 3, the inside of the operation portion 5 and the inside of the universal cord 6 and connected to the light source portion 12 of the peripheral unit 10. Light emitted from the light source portion 12 is guided by the light guide 42 and radiated to an object from an illumination window 47 of the distal end portion (the distal end constituting portion 21) of the insertion portion 3 shown in FIG. 1.

As shown in FIG. 4, the base portion 27 is provided with a support member 51 formed by metal. A proximal end portion of the second flexible tube portion 25 is coupled with a distal end portion of the support member 51.

A distal end portion of the third flexible tube portion 26 is coupled with a proximal end portion of the support member 51. Thereby, the second flexible tube portion 25 and the third flexible tube portion 26 are connected via the base portion 27.

As shown in FIGS. 4 and 5, a cavity portion 52 is defined by the support member 51 in the base portion 27. A driving force transmitting unit 53 is fitted to the support member 51.

The driving force transmitting unit 53 is disposed in the cavity portion 52. The driving force transmitting unit 53 is driven by rotational driving force to cause the rotation unit 30 to rotate being transmitted. The driving force transmitting unit 53 is provided with a driving gear 55.

The driving force transmitting unit 53 is provided with a rotational cylindrical member 58. The rotational cylindrical member 58 is fitted to the base portion 27 in a state in which the support member 51 is inserted in the rotational cylindrical member 58. The rotational cylindrical member 58 is rotatable around the longitudinal axis X relative to the insertion portion 3 (the base portion 27).

Here, two directions in which the rotation unit 30 rotates are assumed to be directions around the longitudinal axis X. On an inner circumferential surface of the rotational cylindrical member 58, an inner circumferential gear portion 59 is provided over the entire circumference in the directions around the longitudinal axis X. The inner circumferential gear portion 59 engages with the driving gear 55.

In the present embodiment, three inner-side rollers 61A to 61C are fitted to the rotational cylindrical member 58. The inner-side rollers 61A to 61C are arranged being separated from one another by a predetermined interval in the direction around the longitudinal axis X.

The inner-side rollers 61A to 61C have corresponding roller shafts Q1 to Q3, respectively. The inner-side rollers 61A to 61C are rotatable relative to the rotational cylindrical member 58 with the corresponding roller shafts Q1 to Q3 as centers, respectively.

Each of the inner-side rollers 61A to 61C is rotatable around the longitudinal axis relative to the insertion portion 3 (the base portion 27) together with the rotational cylindrical member 58.

The outer circumferential direction side of the rotational cylindrical member 58 and the inner-side rollers 61A to 61C is covered with a cover member 62 in a cylindrical shape. A distal end of the cover member 62 is fixed to an outer circumferential surface of the support member 51 via an adhesive portion 63A such as adhesive, and a proximal end of the cover member 62 is fixed to the outer circumferential surface of the support member 51 via an adhesive portion 63B such as adhesive.

The cavity portion 52 in which the driving force transmitting unit 53 is disposed is partitioned from an outside of the insertion portion 3 with the cover member 62. At a position of fixation of the distal end of the cover member 62 and a position of fixation of the proximal end of the cover member 62, watertightness is maintained between the support member 51 and the cover member 62.

Thereby, flow of liquid into the cavity portion 52 and the driving force transmitting unit 53 from the outside of the insertion portion 3 is prevented. At parts where the inner-side rollers 61A to 61C are located, the cover member 62 projects in the outer circumferential direction, in the direction around the longitudinal axis X.

Note that the cover member 62 is fixed relative to the insertion portion 3, and each of the rotational cylindrical member 58 and the inner-side rollers 61A to 61C is rotatable around the longitudinal axis X relative to the cover member 62.

On an inner circumferential surface of the proximal end side cylindrical portion 36, six outer-side rollers 65A to 65F are fitted as shown in FIG. 5. The outer-side rollers 65A to 65F are located on the outer circumferential direction side of the cover member 62.

In the state that the rotation unit 30 is fitted to the insertion portion 3, the inner-side roller 61A is located between the outer-side roller 65A and the outer-side roller 65B, and the inner-side roller 61B is located between the outer-side roller 65C and the outer-side roller 65D, in the direction around the longitudinal axis X.

Furthermore, the inner-side roller 61C is located between the outer-side roller 65E and the outer-side roller 65F in the direction around the longitudinal axis X. The outer-side rollers 65A to 65F have corresponding roller shafts P1 to P6, respectively.

The outer-side rollers 65A to 65F are rotatable relative to the cover member 62 and the proximal end side cylindrical portion 36 with the corresponding roller shafts P1 to P6 as centers, respectively. Each of the outer-side rollers 65A to 65F are rotatable around the longitudinal axis X relative to the insertion portion 3 (the base portion 27) together with the rotation unit 30.

By such a configuration being made, the rotational cylindrical member 58 rotates around the longitudinal axis X when the driving force transmitting unit 53 is driven by rotational driving force. Thereby, the inner-side roller 61A presses the outer-side roller 65A or the outer-side roller 65B.

Similarly, the inner-side roller 61B presses the outer-side roller 65C or the outer-side roller 65D, and the inner-side roller 61C presses the outer-side roller 65E or the outer-side roller 65F.

Thereby, driving force is transmitted from the inner-side rollers 61A to 61C to the outer-side rollers 65A to 65F of the rotation unit 30, and the rotation unit 30 rotates relative to the insertion portion 3 and the cover member 62 with the longitudinal axis X as the center.

As described above, the outer-side rollers 65A to 65F fitted to the proximal end side cylindrical portion 36 constitute a driving force receiving portion configured to receive rotational driving force from the driving force transmitting unit 53 which has been driven.

The outer-side rollers 65A to 65F, which are the driving force receiving portion, are provided on a more proximal end direction side than the spiral tube 31. In the state in which the rotation unit 30 is fitted to the insertion portion 3, the outer-side rollers 65A to 65F are located on the outer circumferential direction side of the base portion 27.

Note that since the inner-side rollers 61A to 61C rotate with the corresponding roller shafts Q1 to Q3 as centers, respectively, friction between each of the inner-side rollers 61A to 61C and the cover member 62 is small.

Similarly, since the outer-side rollers 65A to 65F rotate with the corresponding roller shafts P1 to P6 as centers, respectively, friction between each of the outer-side rollers 65A to 65F and the cover member 62 is small.

Therefore, rotational driving force is appropriately transmitted from the inner-side rollers 61A to 61C to the rotation unit 30, and the rotation unit 30 appropriately rotates.

Note that the proximal end side cylindrical portion 36 is provided with an engaging claw 67 projecting in the inner circumferential direction. The support member 51 of the base portion 27 is provided with an engaged groove 68 over the entire circumference in the direction around the longitudinal axis.

By the engaging claw 67 engaging with the engaged groove 68, movement of the rotation unit 30 along the longitudinal axis X relative to the insertion portion 3 is restricted. In the state in which the engaging claw 67 engages with the engaged groove 68, however, the engaging claw 67 is movable in the direction around the longitudinal axis relative to the engaged groove 68.

As shown in FIGS. 2 and 4, a guide tube 77 is provided being extended along the longitudinal axis X, inside the third flexible tube portion 26 of the insertion portion 3. A distal end of the guide tube 77 is connected to the support member 51 of the base portion 27.

Inside the guide tube 77, a guide channel 78 is formed. A distal end of the guide channel 78 communicates with the cavity portion 52. In the guide channel 78, a driving shaft 79, which is a linear portion, is provided being extended along a shaft axis S.

Rotational driving force generated by the motor 72 is transmitted to the driving shaft 79 via the relay gear 75 and the driving gear 76. By the rotational driving force being transmitted to the driving shaft 79, the driving shaft 79 rotates with the shaft axis S as a center.

A distal end of the driving shaft 79 is connected to the driving gear 55 of the driving force transmitting unit 53. By the driving shaft 79 rotating, rotational driving force is transmitted to the driving force transmitting unit 53, and the driving force transmitting unit 53 is driven. Then, by the rotational driving force being transmitted to the rotational cylindrical member 58, the rotational driving force is transmitted to the rotation unit 30 as described before. Thereby, the rotation unit 30 rotates.

Note that as shown in FIG. 5, bending wires 38A and 38B are provided being extended along the longitudinal axis X inside the insertion portion 3. Inside the operation portion 5, proximal ends of the bending wires 38A and 38B are connected to a pulley (not shown) coupled with the bending operation knob 37.

Distal ends of the bending wires 38A and 38B are connected to a distal end portion of the bending portion 22. By a bending operation on the bending operation knob 37, the bending wire 38A or the bending wire 38B is pulled, and the bending portion 22 bends. Note that, in the present embodiment, the bending portion 22 is configured only with a positive bending portion which bends by a bending operation.

The bending wires 38A and 38B are inserted in corresponding coils 39A and 39B, respectively. Proximal ends of the coils 39A and 39B are provided being extended up to the inside of the operation portion 5. Distal ends of the coils 39A and 39B are connected to an inner circumferential surface of a distal end portion of the first flexible tube portion 23. Note that though the two bending wires 38A and 38B are provided so that the bending portion 22 is bendable in two directions in the present embodiment, for example, four bending wires may be provided so that the bending portion 22 is bendable in four directions.

In the endoscope 2 of the present embodiment, each of the first flexible tube portion 23 and the second flexible tube portion 25 is formed by a first helical tube 91 which is a first flex tube, a first flexible mesh tube 92 which is a first flexible braid tube, and a first flexible outer cover 93 which is an outer cover tube as shown in FIG. 6.

The first helical tube 91, the first flexible mesh tube 92 and the first flexible outer cover 93 are provided being extended along the longitudinal axis X from a distal end of the first flexible tube portion 23 up to a proximal end of the second flexible tube portion 25.

The outer circumferential direction side of the first helical tube 91 is covered with the first flexible mesh tube 92, and the outer circumferential direction side of the first flexible mesh tube 92 is covered with the first flexible outer cover 93.

The first helical tube 91 is provided with a belt-shaped member 95 made of metal. On the first helical tube 91, the belt-shaped member 95 is provided being extended in a helical shape around the longitudinal axis X.

The first flexible mesh tube 92 is provided with metallic strands 96. The first flexible mesh tube 92 is formed by the strands 96 which are woven. The first flexible outer cover 93 is formed by resin material.

A proximal end portion of the bending tube 81 engages with a connecting tube 84 in a cylindrical shape (see FIG. 3), and the first helical tube 91 and the first flexible mesh tube 92 engage with the connecting tube 84 in a state of being inserted on the inner circumferential direction side of the connecting tube 84.

The first flexible outer cover 93 is glued to the bending outer cover 85 via an adhesive portion 86 such as adhesive. The first flexible tube portion 23 and the bending portion 22 are coupled with each other as described before. The first helical tube 91, the first flexible mesh tube 92 and the first flexible outer cover 93 engage with the support member 51 in a state of being inserted on the inner circumferential direction side of the support member 51 as shown in FIG. 4.

Thereby, the second flexible tube portion 25 is coupled with the base portion 27. In the present embodiment, the first helical tube 91, the first flexible mesh tube 92 and the first flexible outer cover 93 are continuously provided being extended between the first flexible tube portion 23 and the second flexible tube portion 25.

Note that the third flexible tube portion 26 is formed by a second helical tube 101 which is a second flex tube, a second flexible mesh tube 102 which is a second flexible braid tube and a second flexible outer cover 103 (reference numerals in parentheses in FIG. 6).

The second helical tube 101, the second flexible mesh tube 102 and the second flexible outer cover 103 are provided being extended along the longitudinal axis X from a distal end of the third flexible tube portion 26 up to a proximal end of the third flexible tube portion 26. The outer circumferential direction side of the second helical tube 101 is covered with the second flexible mesh tube 102, and the outer circumferential direction side of the second flexible mesh tube 102 is covered with the second flexible outer cover 103.

The proximal end portion of the support member 51 engages with a connecting member 104. The second helical tube 101 and the second flexible mesh tube 102 engage with the connecting member 104 in a state of being inserted on the inner circumferential direction side of the connecting member 104 (see FIG. 4). Thereby, the third flexible tube portion 26 is coupled with the base portion 27.

On the second helical tube 101, a belt-shaped member 105 made of metal is provided being extended in a helical shape with the longitudinal axis X as the center.

The second flexible mesh tube 102 is formed by metallic strands 106 which are woven. The second flexible outer cover 103 is formed by resin material.

Here, various configurations of the spiral tube 31 will be described in detail below.

### (First Form of Spiral Tube)

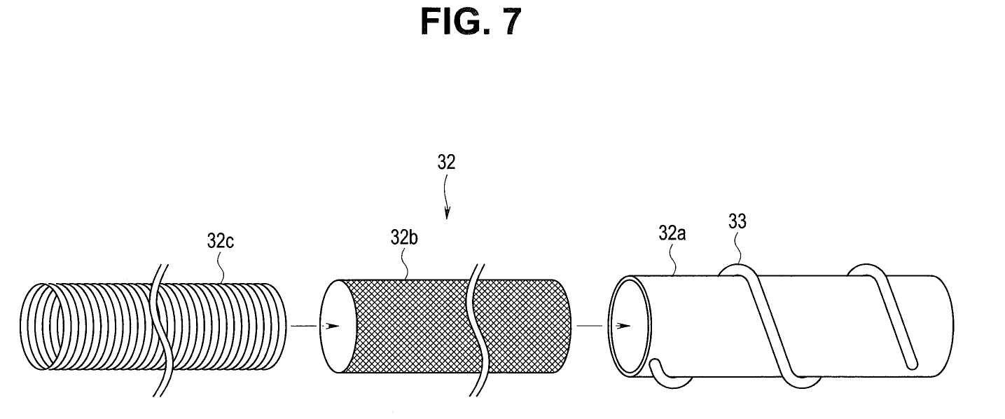

A first form of a configuration of the tube portion 32 which occupies most of the spiral tube 31 will be described below based on FIGS. 7 to 11.

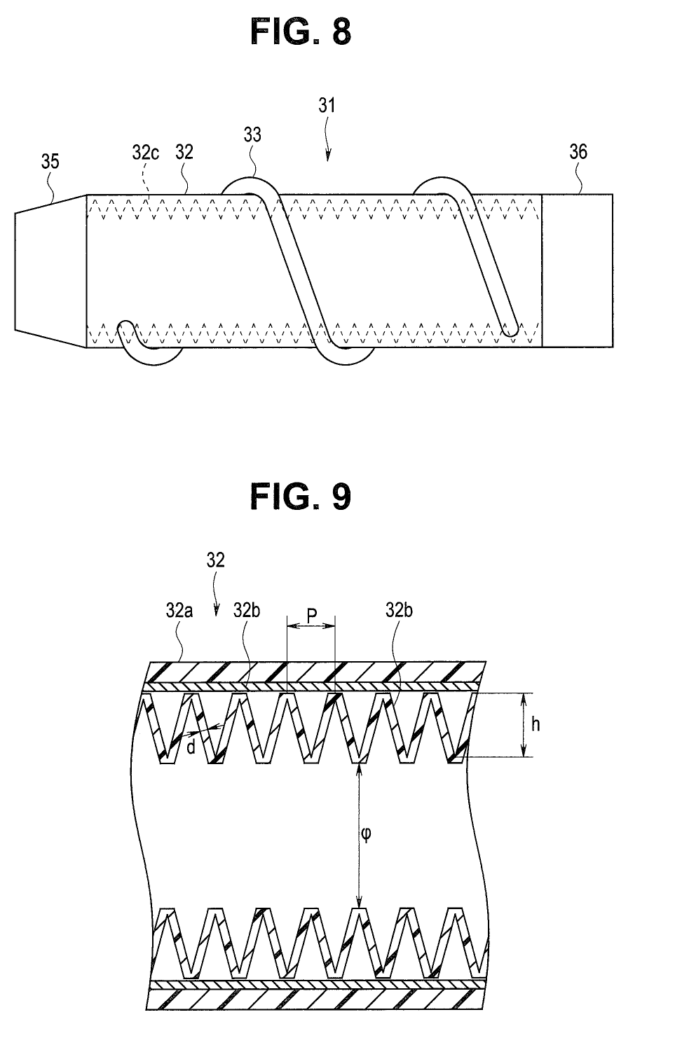

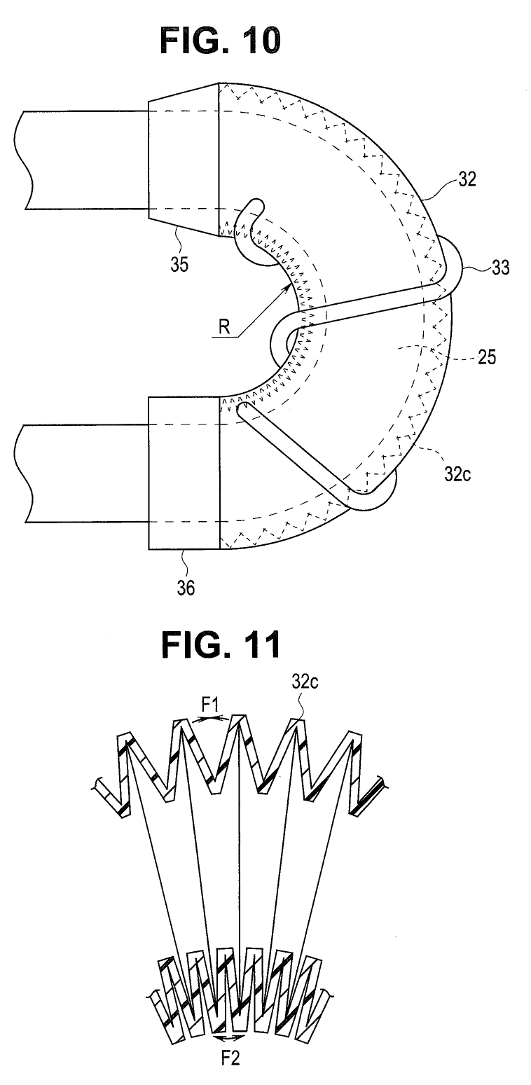

Note that FIG. 7 is an exploded perspective view showing a first form of the spiral tube in which the tube portion is disassembled in members; FIG. 8 is a side view showing the rotation unit; FIG. 9 is a cross-sectional view of the tube portion; FIG. 10 is a side view showing a state in which the insertion portion provided with the rotation unit is bent; and FIG. 11 is a cross-sectional view of a corrugated tube which is bent.

As shown in FIGS. 7 and 8, the tube portion 32 which occupies most of the spiral tube 31 of the present form is provided with a cover tube 32a to be an outer layer, a flexible mesh tube 32b to be a middle layer and a corrugated tube 32c to be an inner layer.

The outer circumferential side of the corrugated tube 32c of the tube portion 32 is covered with the flexible mesh tube 32b, and the outer circumferential side of the flexible mesh tube 32b is covered with the cover tube 32a provided with the fin portion 33.

The flexible mesh tube 32b is a metallic mesh tube formed by metallic strands which are woven. Note that, instead of the flexible mesh tube 32b, an elastic tube may be used for the tube portion 32. The corrugated tube 32c is a so-called pleated flexible tube.

Bending rigidity of the tube portion 32 as a whole is set by the cover tube 32a, the flexible mesh tube 32b and the corrugated tube 32c.

More specifically, in addition to predetermined bending rigidity of the cover tube 32a and the flexible mesh tube 32b, predetermined bending rigidity by the corrugated tube 32c is set for the tube portion 32 of the present form.

As shown in FIG. 9, the bending rigidity of the corrugated tube 32c is decided by various parameters (components by structures of various members) such as a pitch P, thickness d, height h of unevenness, inner diameter ϕ and material.

FIG. 10 shows a state in which the spiral tube 31 is bent at an arbitrary bending angle R, at 180° here. At this time, on the corrugated tube 32c, a sum (F1+F2) of a bending stress F1 due to tensile force generated on an outer bending side, which is force of the corrugated tube trying to return to a linear state due to bending rigidity per pitch P between tops, and a bending stress F2 due to repulsive force generated on an inner bending side is generated as shown in FIG. 11.

For the whole corrugated tube 32c, a stress corresponding to a product {nP×(F1+F2)} of the number (n) of pitches P and the bending stress (F1+F2) per pitch P is generated, and bending rigidity is decided.

Thus, for the spiral tube 31, the predetermined bending rigidity in the state in which the spiral tube 31 is bent at the arbitrary bending angle R, for example, at 180° here is set by the predetermined bending rigidity of the cover tube 32a and the flexible mesh tube 32b and the predetermined bending rigidity by the corrugated tube 32c, and the predetermined bending rigidity is decided by the various parameters (components by structures of various members) described above.

Note that the arbitrary bending angle R is not limited to 180° but can be appropriately set to a predetermined angle at which the spiral tube 31 rotates without stopping relative to rotational torque (driving torque) by the motor 72 which is a driving source.

### (Second Form of Spiral Tube)

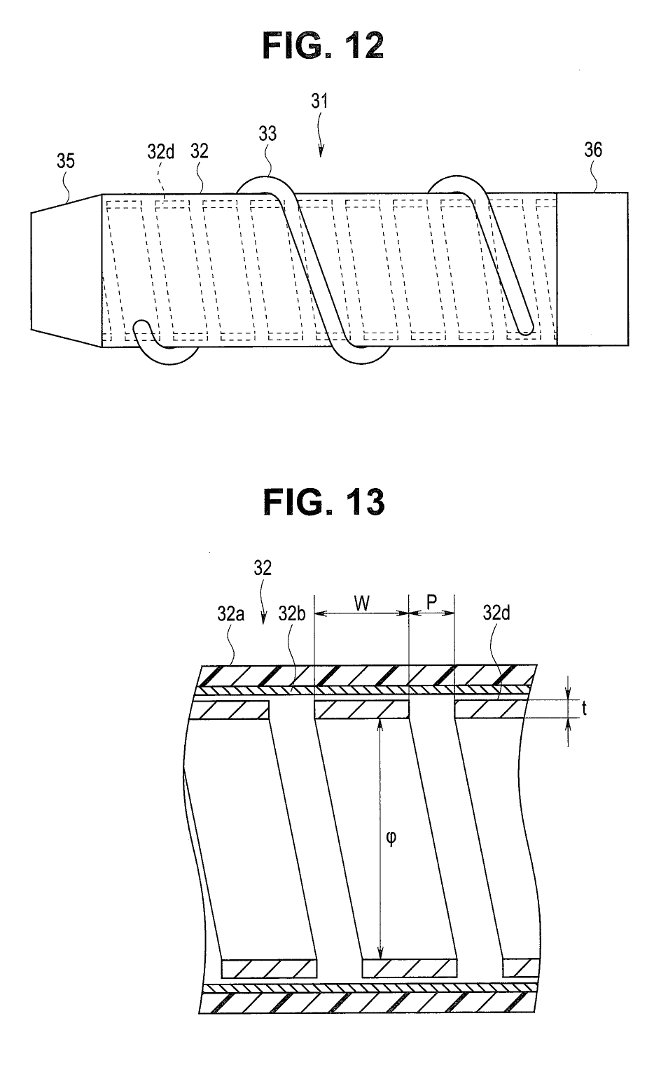

A second form of the configuration of the tube portion 32 which occupies most of the spiral tube 31 will be described below based on FIGS. 12 to 15.

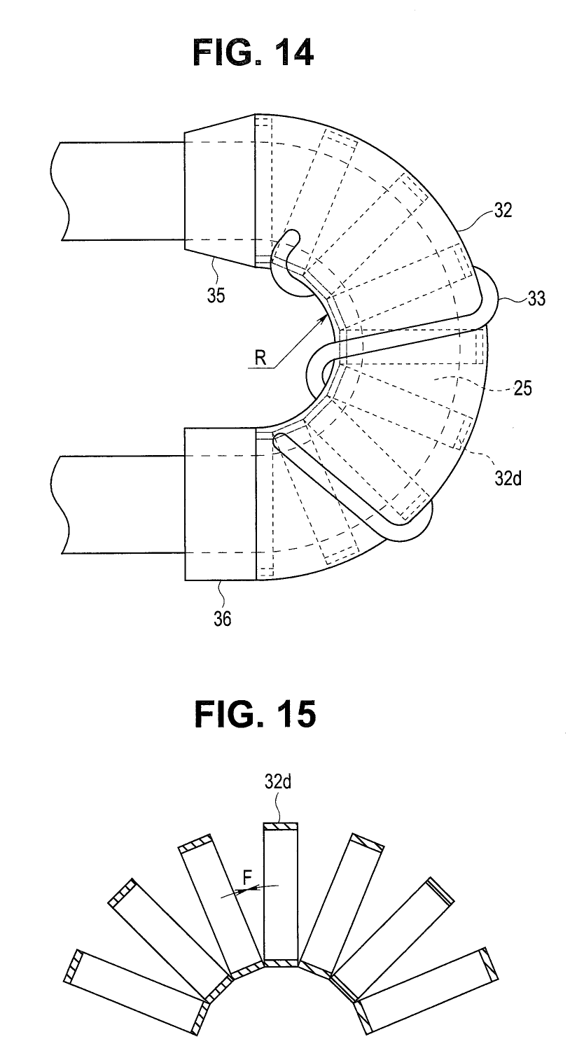

Note that FIG. 12 is a side view showing a second form of the spiral tube and showing the rotation unit; FIG. 13 is a cross-sectional view of the tube portion; FIG. 14 is a side view showing a state in which the insertion portion provided with the rotation unit is bent; and FIG. 15 is a cross-sectional view of the helical tube which is bent.

As shown in FIGS. 12 and 13, the tube portion 32 which occupies most of the spiral tube 31 of the present form is provided with the cover tube 32a to be an outer layer, the flexible mesh tube 32b to be a middle layer and, here, a helical tube 32d to be an inner layer instead of the corrugated tube 32c.

The outer circumferential side of the helical tube 32d of the tube portion 32 is covered with the flexible mesh tube 32b, and the outer circumferential side of the flexible mesh tube 32b is covered with the cover tube 32a provided with the fin portion 33. The helical tube 32d is a tube body having flexibility, which is formed by helically winding a belt-shaped member made of metal.

The bending rigidity of the whole tube portion 32 is set by the cover tube 32a, the flexible mesh tube 32b and the helical tube 32d.

More specifically, in addition to the predetermined bending rigidity of the cover tube 32a and the flexible mesh tube 32b, predetermined bending rigidity by the helical tube 32d is set for the tube portion 32 of the present form.

As shown in FIG. 9, the bending rigidity of the helical tube 32d is decided by various parameters (components by structures of various members) such as a pitch P, width W, thickness t, inner diameter ϕ and material of the belt-shaped member to be wound.

FIG. 14 shows a state in which the spiral tube 31 is bent at the arbitrary bending angle R, for example, at 180° here. At this time, on the helical tube 32d, such a bending stress F due to tensile force generated on an outer bending side that the helical tube tries to return to a linear state due to bending rigidity per pitch P of the belt-shaped member to be wound is generated as shown in FIG. 15.

For the whole helical tube 32d, a stress corresponding to a product (nP×F) of the number (n) of pitches P and the bending stress (F) per pitch P is generated, and bending rigidity is decided.

Thus, for the spiral tube 31, the predetermined bending rigidity in the state in which the spiral tube 31 is bent at the arbitrary bending angle R, at 180° here is set by the predetermined bending rigidity of the cover tube 32a and the flexible mesh tube 32b and the predetermined bending rigidity by the helical tube 32d, and the predetermined bending rigidity is decided by the various parameters (components by structures of various members) described above.

Note that the arbitrary bending angle R is not limited to 180° but can be appropriately set to a predetermined angle at which the spiral tube 31 rotates without stopping relative to rotational torque (driving torque) by the motor 72 which is a driving source.

### (Third Form of Spiral Tube)

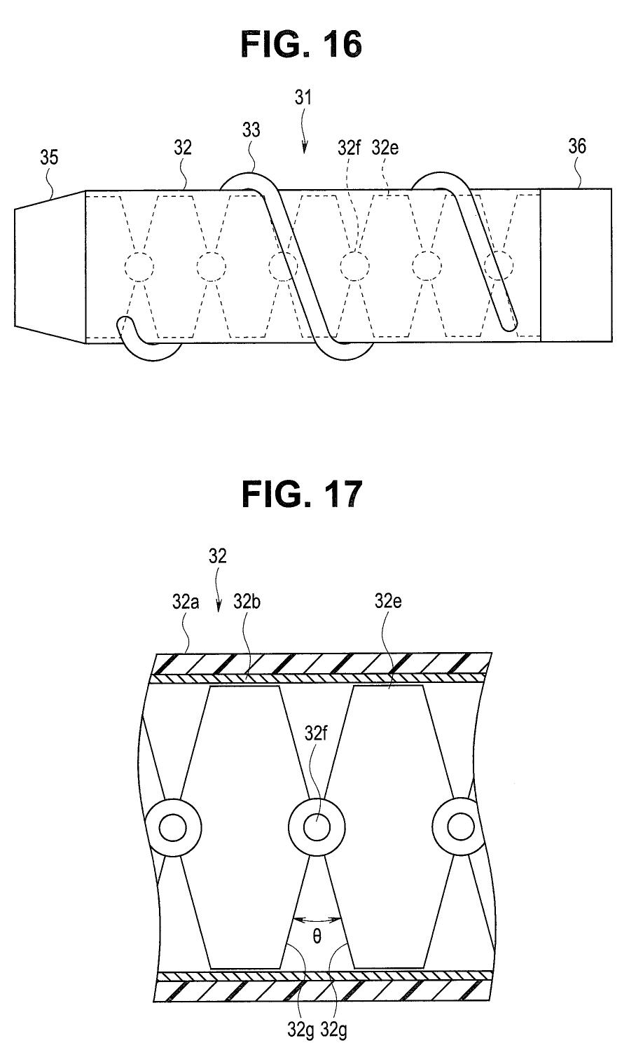

A third form of the configuration of the tube portion 32 which occupies most of the spiral tube 31 will be described below based on FIGS. 16 to 18.

Note that FIG. 16 is a side view showing the third form of the spiral tube and showing the rotation unit; FIG. 17 is a cross-sectional view of the tube portion; FIG. 14 is a side view showing a state in which the insertion portion provided with the rotation unit is bent.

As shown in FIGS. 16 and 17, the tube portion 32 which occupies most of the spiral tube 31 of the present form is provided with the cover tube 32a to be an outer layer, the flexible mesh tube 32b to be a middle layer and, here, a plurality of bending restricting pieces 32e instead of the corrugated tube 32c or the helical tube 32d.

The outer circumferential side of the plurality of bending restricting pieces 32e of the tube portion 32 is covered with the flexible mesh tube 32b, and the outer circumferential side of the flexible mesh tube 32b is covered with the cover tube 32a provided with the fin portion 33. The plurality of bending restricting pieces 32e are rotatably coupled by pivoting portions 32f such as rivets to constitute a bending tube.

Here, a bending state of the whole tube portion 32 is restricted by the plurality of bending restricting pieces 32e. The bending angle R is specified by facing end faces 32g of the plurality of bending restricting pieces 32e coming into contact with each other, and decided by an angle θ formed by two facing end faces 32g in the linear state.

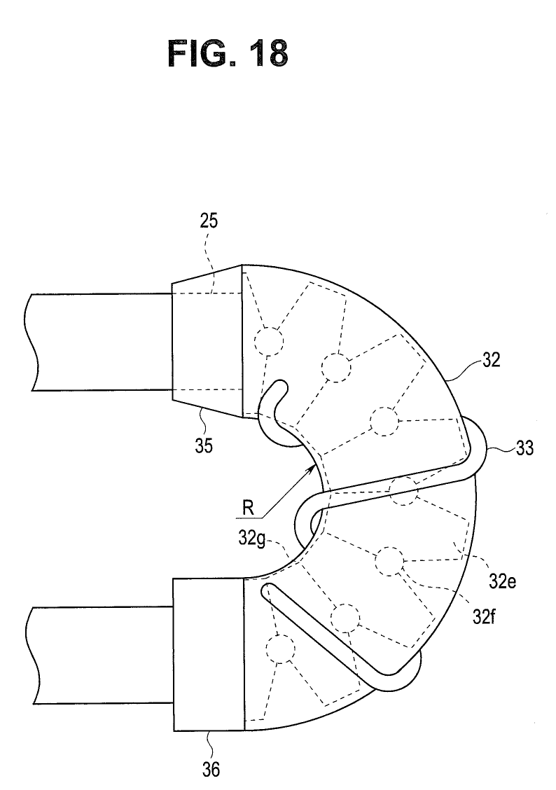

FIG. 18 shows a state in which the spiral tube 31 is bent at the arbitrary bending angle R, for example, at 180° here. At this time, facing end faces 32g of the plurality of bending restricting pieces 32e on the inner bending side come into contact with each other, and a maximum bending angle R is specified.

That is, the bending angle R of the spiral tube 31 is decided by a shape of the plurality of bending restricting pieces 32e. For example, when two adjoining bending restricting pieces 32e are assumed to be one set (one pair), the bending angle R of the spiral tube 31 is decided by a product of a bending angle of the one set of the bending restricting pieces 32e and the number of the pivoting portions 32f.

Note that though a configuration in which the bending tube formed by the plurality of coupled bending restricting pieces 32e is bent in two directions is shown here, a configuration may be, of course, adopted in which the coupling positions by the pivoting portions 32f are changed in a circumferential direction so that three-dimensional bending is possible.

Note that the arbitrary bending angle R is not limited to 180° but can be appropriately set to a predetermined angle at which the spiral tube 31 rotates without stopping relative to rotational torque (driving torque) by the motor 72 which is a driving source.

Next, various configurations of the second flexible tube portion 25 as a part of the insertion portion 3 where the rotation unit 30 is fitted on the outer circumferential side and the spiral tube 31 is fitted will be described in detail below.

### (First Form of Second Flexible Tube Portion)

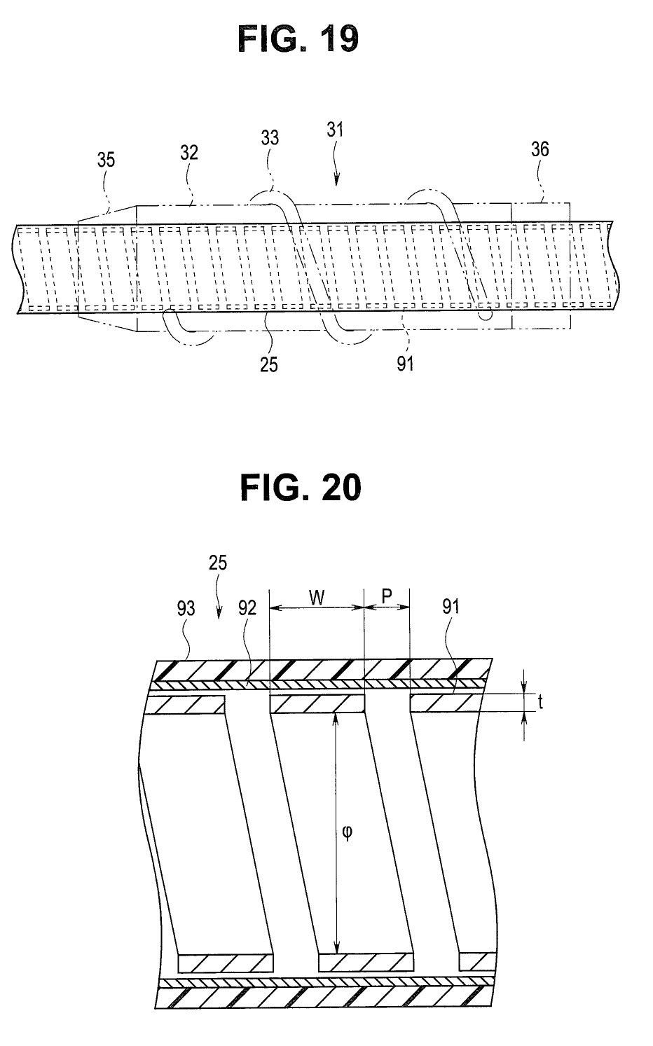

A first form of a configuration of the second flexible tube portion 25 will be described below based on FIGS. 19 to 22.

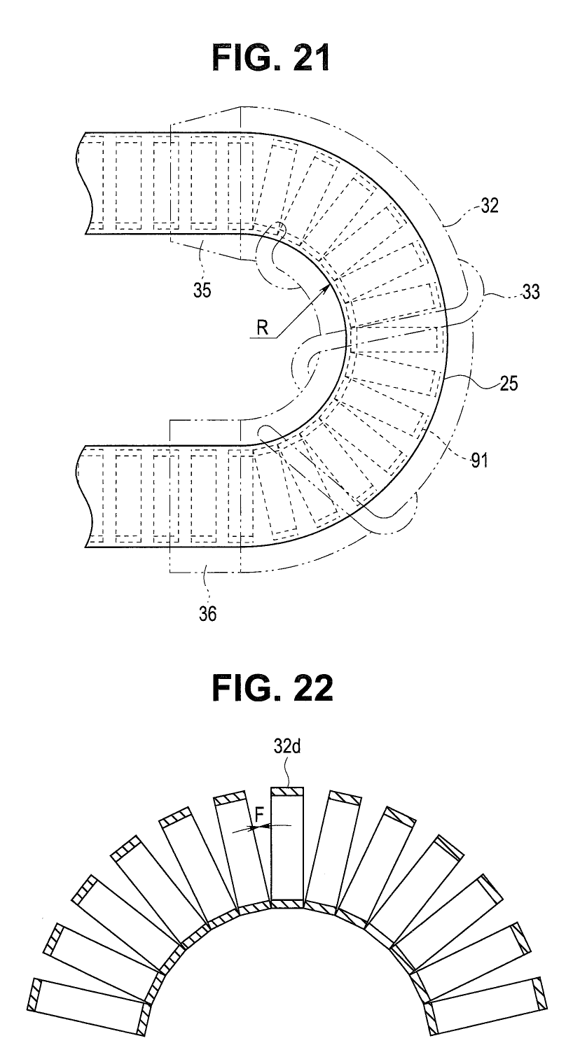

Note that FIG. 19 is a side view showing the first form of the second flexible tube portion and showing the second flexible tube portion equipped with the rotation unit; FIG. 20 is a cross-sectional view of the second flexible tube portion; FIG. 21 is a side view showing a state in which the insertion portion provided with the rotation unit is bent; and FIG. 22 is a cross-sectional view of the helical tube which is bent.

As shown in FIGS. 19 and 20 and described before, the second flexible tube portion 25 as the part of the insertion portion 3 where the spiral tube 31 of the present form is fitted is configured having the first helical tube 91 which is the first flex tube, the first flexible mesh tube 92 as a covering layer, which is the first flexible braid tube, and the first flexible outer cover 93 as a coating layer, which is the outer cover tube.

The outer circumferential side of the first helical tube 91 of the second flexible tube portion 25 is covered with the first flexible mesh tube 92, and the outer circumferential side of the first flexible mesh tube 92 is covered with the first flexible outer cover 93. Note that an elastic tube may be used for the first flexible mesh tube 92.

The first helical tube 91 is a tube body having flexibility, which is formed by helically winding a belt-shaped member made of metal. Bending rigidity of the whole second flexible tube portion 25 is set by the first helical tube 91, the first flexible mesh tube 92 and the first flexible outer cover 93.

More specifically, for the second flexible tube portion 25 of the present form, predetermined bending rigidity by the first helical tube 91 is set in addition to predetermined bending rigidity of the first flexible outer cover 93 and the first flexible mesh tube 92 and bending rigidity of various internal components such as the image pickup cable 41, the light guide 42 and the channel tube 43.

As shown in FIG. 20, the bending rigidity of the first helical tube 91 is decided by various parameters (components by structures of various members) such as a pitch P, width W, thickness t, inner diameter 4) and material of the belt-shaped member to be wound.

FIG. 21 shows a state in which the first helical tube 91 equipped with the spiral tube 31 of the rotation unit 30 is bent at the arbitrary bending angle R, for example, at 180° here. At this time, on the first helical tube 91, such a bending stress F due to tensile force generated on the outer bending side that the first helical tube tries to return to a linear state due to bending rigidity per pitch P of the belt-shaped member to be wound is generated as shown in FIG. 22.

For the whole first helical tube 91, a stress corresponding to a product (nP×F) of the number (n) of pitches P and the bending stress (F) per pitch P is generated, and bending rigidity is decided.

Thus, for the second flexible tube portion 25, the predetermined bending rigidity in the state in which the second flexible tube portion 25 is bent at the arbitrary bending angle R, at 180° here is set by the predetermined bending rigidity of the first flexible outer cover 93 and the first flexible mesh tube 92 and the predetermined bending rigidity by the first helical tube 91, and the predetermined bending rigidity is decided by the various parameters (components by structures of various members) described above.

Note that the arbitrary bending angle R is not limited to 180° but can be appropriately set to a predetermined angle at which the spiral tube 31 rotates without stopping relative to rotational torque (driving torque) by the motor 72 which is a driving source.

### (Second Form of Second Flexible Tube Portion)

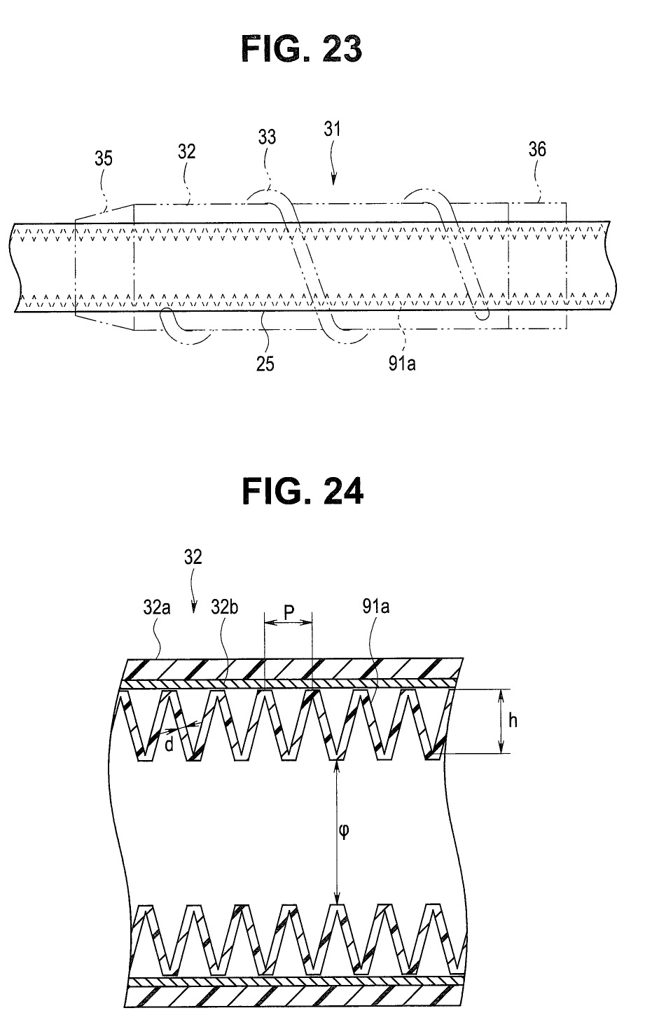

A first form of the configuration of the second flexible tube portion 25 will be described below based on FIGS. 23 to 26.

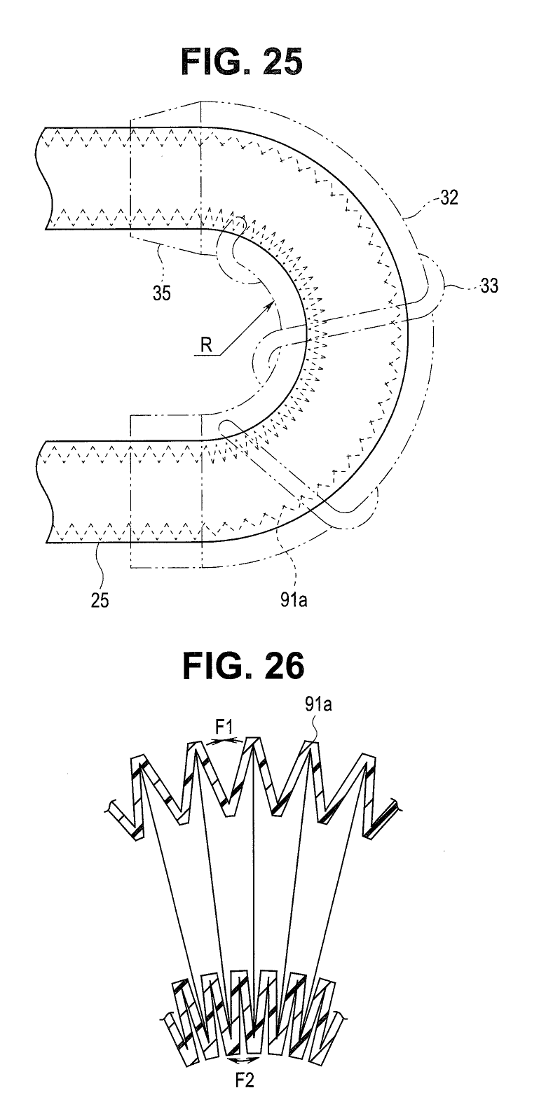

Note that FIG. 23 is a side view showing the second flexible tube portion equipped with the rotation unit of the second form of the spiral tube; FIG. 24 is a cross-sectional view of the second flexible tube portion; FIG. 25 is a side view showing a state in which the second flexible tube portion equipped with the rotation unit is bent; FIG. 26 is a cross-sectional view of the corrugated tube which is bent.

The second flexible tube portion 25 as the part of the insertion portion 3 where the spiral tube 31 of the present form is fitted is shown in FIGS. 23 and 24 and is configured having a corrugated tube 91a instead of the first helical tube 91, the first flexible mesh tube 92 as the covering layer which is the first flexible braid tube, and the first flexible outer cover 93 as the coating layer which is the outer cover tube.

The outer circumferential side of the corrugated tube 91a of the second flexible tube portion 25 is covered with the first flexible mesh tube 92, and the outer circumferential side of the first flexible mesh tube 92 is covered with the first flexible outer cover 93. The first helical tube 91 is a tube body having flexibility, which is formed by helically winding a belt-shaped member made of metal. Note that an elastic tube may be used for the first flexible mesh tube 92. The corrugated tube 91a is a so-called pleated flexible tube.

The bending rigidity of the whole second flexible tube portion 25 is set by the corrugated tube 91a, the first flexible mesh tube 92 and the first flexible outer cover 93.

More specifically, for the second flexible tube portion 25 of the present form, predetermined bending rigidity by the corrugated tube 91a is set in addition to the predetermined bending rigidity of the first flexible mesh tube 92 and the first flexible outer cover 93 and the bending rigidity of various internal components such as the image pickup cable 41, the light guide 42 and the channel tube 43.

As shown in FIG. 24, the bending rigidity of the corrugated tube 91a is decided by various parameters (components by structures of various members) such as a pitch P, thickness d, height h of unevenness, inner diameter ϕ and material.

FIG. 25 shows a state in which the second flexible tube portion 25 is bent at the arbitrary bending angle R, at 180° here. At this time, on the corrugated tube 91a, a sum (F1+F2) of such a bending stress F1 due to tensile force generated on an outer bending side that the corrugated tube tries to return to a linear state due to bending rigidity per pitch P between tops and a bending stress F2 due to repulsive force generated on an inner bending side is generated as shown in FIG. 26.

For the whole corrugated tube 91a, a stress corresponding to a product {nP× (F1+F2)} of the number (n) of pitches P and the bending stress (F1+F2) per pitch P is generated, and bending rigidity is decided.

Thus, for the second flexible tube portion 25, the predetermined bending rigidity in the state in which the second flexible tube portion 25 is bent at the arbitrary bending angle R, for example, at 180° here is set by the predetermined bending rigidity of the first flexible mesh tube 92 and the first flexible outer cover 93 and the predetermined bending rigidity by the corrugated tube 91a, and the predetermined bending rigidity is decided by the various parameters (components by structures of various members) described above.

Note that the arbitrary bending angle R is not limited to 180° but can be appropriately set to a predetermined angle at which the spiral tube 31 rotates without stopping relative to rotational torque (driving torque) by the motor 72 which is a driving source.

### (Third Form of Second Flexible Tube Portion)

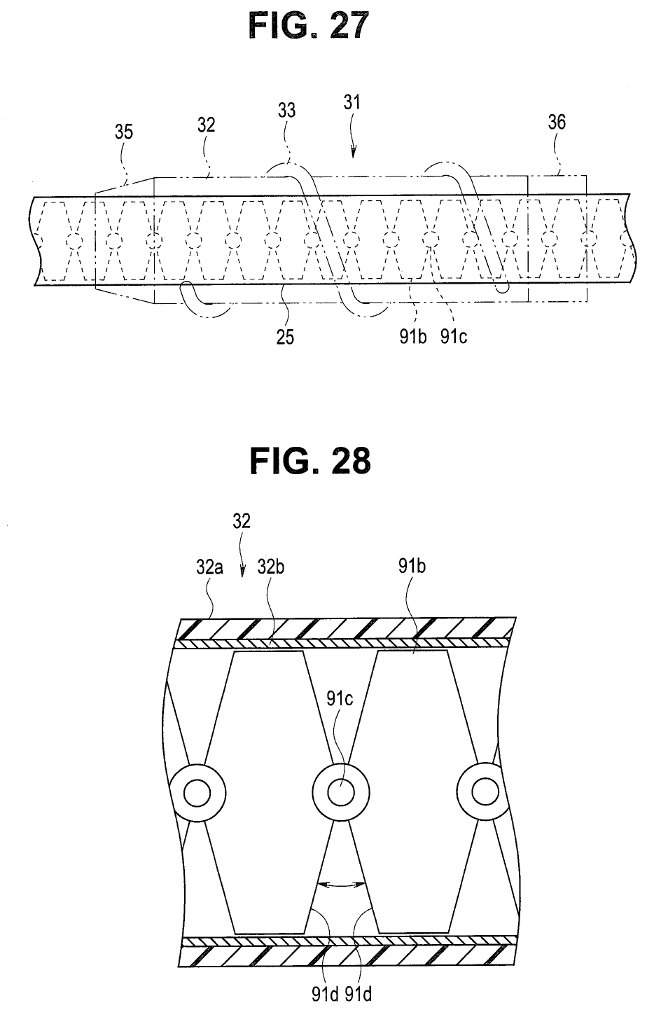

A third form of the configuration of the second flexible tube portion 25 will be described below based on FIGS. 27 to 29.

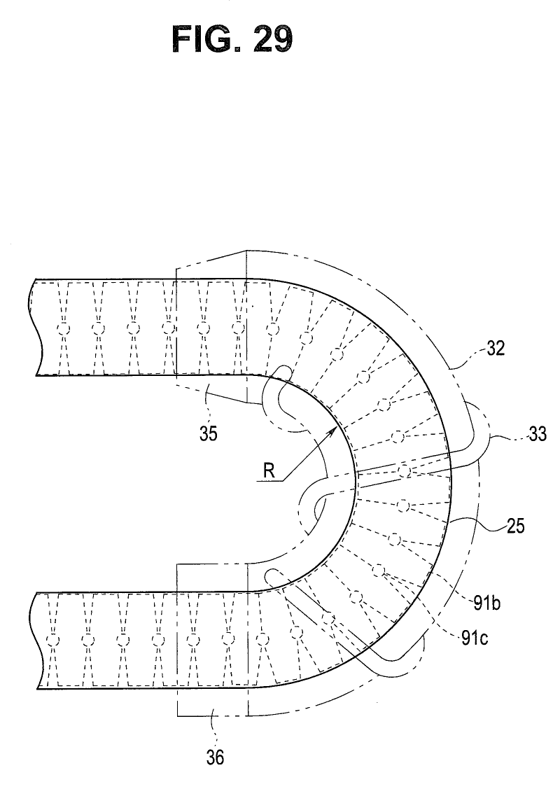

Note that FIG. 27 is a side view showing the third form of the second flexible tube portion and showing the second flexible tube portion equipped with the rotation unit; FIG. 28 is a cross-sectional view of the second flexible tube portion; and FIG. 29 is a side view showing a state in which the second flexible tube portion provided with the rotation unit is bent.

As shown in FIGS. 27 and 28, the second flexible tube portion 25 as the part of the insertion portion 3 where the spiral tube 31 of the present form is fitted is configured having a plurality of bending restricting pieces 91b constituting a bending tube, instead of the first helical tube 91 or the corrugated tube 91a, the first flexible mesh tube 92 as the covering layer which is the first flexible braid tube, and the first flexible outer cover 93 as the coating layer which is the outer cover tube.

The outer circumferential side of the plurality of bending restricting pieces 91b of the second flexible tube portion 25 is covered with the first flexible mesh tube 92, and the outer circumferential side of the first flexible mesh tube 92 is covered with the first flexible outer cover 93. The first helical tube 91 is a tube body having flexibility, which is formed by helically winding a belt-shaped member made of metal. Note that an elastic tube may be used for the first flexible mesh tube 92.

The plurality of bending restricting pieces 91b are rotatably coupled by pivoting portions 91c such as rivets to constitute a bending tube.

Here, a bending state of the whole second flexible tube portion 25 is restricted by the plurality of bending restricting pieces 91b. The bending angle R is specified by facing end faces 91d of the plurality of bending restricting pieces 91b coming into contact with each other, and decided by an angle θ formed by two facing end faces 91d in the linear state.

FIG. 29 shows the state in which the second flexible tube portion 25 is bent at the arbitrary bending angle R, for example, at 180° here. At this time, facing end faces 91d of the plurality of bending restricting pieces 91b on an inner bending side come into contact with each other, and a maximum bending angle R is specified.

That is, the bending angle R of the second flexible tube portion 25 is decided by a shape of the plurality of bending restricting pieces 91b. For example, when two adjoining bending restricting pieces 91b are assumed to be one set (one pair), the bending angle R of the second flexible tube portion 25 is decided by a product of a bending angle of the one set of the bending restricting pieces 91b and the number of the pivoting portions 91c.

Note that though a configuration in which the bending tube formed by the plurality of coupled bending restricting pieces 91b is bent in two directions is shown here, a configuration may be, of course, adopted in which the coupling positions by the pivoting portions 91c are changed in a circumferential direction so that three-dimensional bending is possible.

The endoscope apparatus 1 of the present embodiment configured as described above is an insertion apparatus provided with the rotation unit 30 and the endoscope 2 which is an insertion device. Operation and effects of the endoscope apparatus 1 will be described.

At the time of using the endoscope apparatus 1, the insertion portion 3 and the rotation unit 30 are inserted into a body cavity in a state in which the rotation unit 30 is fitted to the insertion portion 3. Then, by driving the motor 72 in a state in which the fin portion 33 of the spiral tube 31 is in contact with a body cavity wall, rotational driving force is transmitted to the driving force transmitting unit 53 fitted to the base portion 27 of the insertion portion 3.

Then, the driving force transmitting unit 53 is driven, and the outer-side rollers 65A to 65F, which are a driving force receiving portion, receive rotational driving force from the driving force transmitting unit 53. Thereby, the rotation unit 30 rotates with the longitudinal axis X as the center.

By the rotation unit 30 rotating with the longitudinal axis X as the center, in a state in which the fin portion 33 of the spiral tube 31 is pressed in the inner circumferential direction by the body cavity wall or the like, thrust to move the insertion portion 3 forward in the distal end direction or move backward in the proximal end direction acts on the insertion portion 3 and the rotation unit 30.

At this time, in the endoscope apparatus 1 of the present embodiment, when the insertion portion 3 passes through a crooked part of the body cavity, for example, from an oral cavity to a pharynx of an esophagus which is an upper side body cavity, an ileocecal valve of a small intestine existing near a cecum, and, from an anus to a splenic flexure, hepatic flexure and the like of a large intestine which is a lower side body cavity, the spiral tube 31 of the rotation unit 30 does not excessively bend, so that rotation is prevented from stopping.

More specifically, as for driving torque by the motor 72 to drive the rotation unit 30, various transmission losses of driving systems, such as friction losses by gear portions such as the driving gears 55 and 76 and the relay gear 75, friction losses by the driving shaft 79, the guide channel 78 and the like, and friction losses of the inner-side rollers 61A to 61C, the outer-side rollers 65A to 65F and the like relative to the proximal end side cylindrical portion 36 or the cover member 62 occur.

In addition to the transmission losses of the driving systems, rotation losses of friction resistance and the like due to bending of the spiral tube 31 occur. Therefore, by preventing total losses of the transmission losses of the driving systems and the rotation losses due to bending of the spiral tube 31 from exceeding driving torque by the motor 72, it is possible to prevent rotation of the spiral tube 31 of the rotation unit 30 from stopping.

Therefore, in the present embodiment, restrictions on the bending rigidity or the maximum bending angle of the spiral tube 31 and/or the second flexible tube portion 25 of the rotation unit 30 are set so that the spiral tube 31 does not excessively bend, and rotation of the spiral tube 31 is prevented from stopping, as described before.

That is, at the time of the insertion portion 3 being inserted into a body cavity, the spiral tube 31 bends in various shapes according to a shape in which a body cavity runs and movability.

In order to cause the bent spiral tube 31 to smoothly rotate, sufficient driving torque by the motor 72 is required because inner side bending of the bent spiral tube 31 is compressed, and expanding/contracting force due to being pulled, force of friction with the second flexible tube portion 25 and force of friction with the body cavity wall occur on outer side bending.

At this time, when being bent at a large angle (with a small radius of curvature) or three-dimensionally bent, the spiral tube 31 requires driving torque by the motor 72 which is necessary to rotate.

Since the endoscope 2 has a configuration in which the part of the second flexible tube portion 25 equipped with the spiral tube 31 of the rotation unit 30, which is provided on the insertion portion 3, restricts bending rigidity larger than reaction force received as external force when a bending body cavity tries to maintain its shape or a maximum bending angle, according to thrust at the time of coming into contact with the body cavity wall by rotation of the spiral tube 31 and moving forward or backward and force of moving forward or backward generated by the insertion portion 3 being pushed or pulled by a user, it is possible to prevent rotation of the spiral tube 31 from stopping.

Therefore, the endoscope apparatus 1 of the present embodiment is configured to, for the bending rigidity of the tube portion 32 of the spiral tube 31 and/or the bending rigidity of the second flexible tube portion 25, combine various components and set total bending rigidity by a structure of the part of the second flexible tube portion 25 equipped with the spiral tube 31, based on various parameters (components by structures of various members) as described before so that rotation of the spiral tube 31 is prevented from being stopped by predetermined driving torque by the motor 72.

That is, by combining the configuration described in the first or second form, in which the bending rigidity of the tube portion 32 of the spiral tube 31 is set, and the configuration described in the first or second form, in which the bending rigidity of the second flexible tube portion 25 is set, total bending rigidity by the structure of the part of the second flexible tube portion 25 equipped with the spiral tube 31 is set, and, thereby, it is possible to make a configuration in which the spiral tube 31 does not excessively bend and prevent rotation of the spiral tube 31 from stopping.

Note that, by restricting the maximum bending angle of either the spiral tube 31 of the third form or the second flexible tube portion 25 of the third form by the plurality of bending restricting pieces 32e or 91b, the endoscope apparatus 1 can prevent the spiral tube 31 or the second flexible tube portion 25 from bending more, so that the spiral tube 31 does not excessively bend, and rotation of the spiral tube 31 can be prevented from stopping.

That is, in the case of using the plurality of bending restricting pieces 32e or 91b described above, one of the spiral tube 31 and the second flexible tube portion 25 may be in a conventional configuration.

Further, by using the second flexible tube portion 25 in a conventional configuration and using only the configuration described in the first or second form in which the bending rigidity of the tube portion 32 of the spiral tube 31 is set, the total bending rigidity can be set by the structure of the part of the second flexible tube portion 25 equipped with the spiral tube 31.

Furthermore, by using the spiral tube 31 in a conventional configuration and using only the configuration described in the first or second form in which the bending rigidity of the second flexible tube portion 25 is set, the total bending rigidity can be set by the structure of the part of the second flexible tube portion 25 equipped with the spiral tube 31.

According to the content described above, in the endoscope apparatus 1, which is the insertion apparatus of the present embodiment, even if the insertion portion 3 is bent in various shapes according to a crooked state of a body cavity, mobility and the like at the time of inserting the insertion portion 3 into the body cavity, rotation of the spiral tube 31, which is a driven member, does not stop.

Since the endoscope apparatus 1 does not cause rotation of the spiral tube 31 to stop, it is possible to use output of rotational torque (driving torque) generated by the motor 72, which is a driving source, as output similar to conventional output, and it is not necessary to increase the size of the motor 72. Thereby, the endoscope apparatus 1 can prevent increase in the size of the operation portion 5 in which the motor 72 is provided, and weight does not increase, either.

Note that, in the endoscope apparatus 1, it becomes unnecessary to provide a speed reducer or the like for earning rotation torque of the motor 72 in the operation portion 5 or the rotation unit 30.

Therefore, in the endoscope apparatus 1 of the present embodiment enables the spiral tube 31, which is a driven member, to smoothly rotate by predetermined driving force by the motor 72, which is a driving source, and can prevent increase in the diameter of the insertion portion 3 or increase in the size and weight of the operation portion 5.

Note that the present invention is not limited to the embodiment described above, but various modifications can be made within a range not departing from the spirit of the invention.

According to the present invention, it is possible to realize an insertion apparatus which enables a driven member to smoothly rotate by predetermined driving force and prevents increase in the diameter of an insertion portion or increase in the size or weight of an operation portion.

The present invention is not limited to the embodiment described above, but various changes, alterations and the like are possible within a range not changing the spirit of the present invention.

What is claimed is:

1. An insertion apparatus comprising:
an insertion portion which is inserted into a body cavity of a subject and provided with a cylindrical body rotatable around a longitudinal axis on an outer circumferential surface, the insertion portion having predetermined flexibility; and
a driving source configured to cause the cylindrical body to rotate; wherein
the cylindrical body is configured with a structure, the structure as a whole having predetermined bending rigidity and the structure being set not to bend at an arbitrary bending angle or more so that rotation by driving force of the driving source does not stop even if the structure receives external force to try to maintain a bending shape of the body cavity from a body cavity wall that the structure touches, due to the predetermined bending rigidity.

2. The insertion apparatus according to claim 1, wherein
the cylindrical body is detachably provided on the outer circumferential surface of the insertion portion.

3. The insertion apparatus according to claim 1, wherein
the cylindrical body is a spiral tube including a helical fin inclined relative to the longitudinal axis, on an outer circumferential surface.

4. The insertion apparatus according to claim 1, wherein
a corrugated tube is included inside the cylindrical body.

5. The insertion apparatus according to claim 1, wherein
a helical tube is included inside the cylindrical body.

6. The insertion apparatus according to claim 1, wherein
a plurality of bending restricting pieces are included inside the cylindrical body and the cylindrical body is set not to bend at the arbitrary angle or more.
